(19) United States
(12) Patent Application Publication
(10) Pub. No.: US 2014/0336338 A1
(43) Pub. Date: Nov. 13, 2014

(54) NANOFILTRATION PROCESS WITH PRE-TREATMENT TO ENHANCE SOLUTE FLUX

(71) Applicant: DUPONT NUTRITION BIOSCIENCES APS, 100 Copenhagen K (DK)

(72) Inventors: Jari Mattila, Kirkkonummi (FI); Hannu Koivikko, Kantvik (FI)

(21) Appl. No.: 14/363,271

(22) PCT Filed: Dec. 5, 2012
(86) PCT No.: PCT/EP2012/074490

§ 371 Date: Jun. 5, 2014

Related U.S. Application Data
(60) Provisional application No. 61/567,815, filed on Dec. 7, 2011.

(51) Int. Cl. B01D 67/00; C07H 1/06; B01D 61/02

(52) U.S. Cl. CPC B01D 67/0093; B01D 61/027; C07H 1/06
USPC 525/435; 210/638; 536/127

(57) ABSTRACT

A process of treating polymeric nanofiltration membranes before separation of low molecular weight compounds from a solution comprising the same by nanofiltration, wherein the treatment of the nanofiltration membranes is performed with an treatment liquid under conditions which enhance the flux of the low molecular weight compounds to the nanofiltration permeate.

### FIELD OF THE INVENTION

The invention relates to a process of treating polymeric nanofiltration membranes, especially membranes selected from polyamide membranes. The process of the invention is based on treating the membranes with treatment liquids, which contain compounds selected from organic acids and alcohols, organic sulfonic acids and sulfonates, surfactants and weak bases, even at very low concentrations and at high temperatures for a prolonged time before their use in nanofiltration. It has been surprisingly found that the treatment process of the invention provides an improved throughput capacity, which remains at a high level in long term in successive nanofiltration cycles, while improving or essentially retaining the separation efficiency of the nanofiltration.

### BACKGROUND OF THE INVENTION

It is generally known in the art that various post-treatment methods are used by the manufacturers of nanofiltration membranes to increase the performance of asymmetric composite membranes and to stabilize the membranes in the longer term, see Nanofiltration—Principles and Applications, edited by A. I. Schafer, A. G. Fane & T. D. Waite, 2005, pages 41-42 (3.2.7 Post treatment). The post-treatment may comprise annealing in water or under dry conditions, exposure to concentrated mineral acids, drying with solvent exchange techniques and treatment with conditioning agents. As useful solvent systems for asymmetric polyimide membranes in the solvent exchange techniques, a combination of isopropanol or methylketone with hexane as well as mixtures of lube oil, methylketone and toluene are specifically mentioned. It is also recited that conservation in conditioning agents, like lube oil, enhances the performance of asymmetric polyimide membranes. The post-treatment for the polyimide membranes in accordance with the cited reference is performed to improve the hydrophilic properties of the membranes.

Furthermore, the same textbook as mentioned above describes fouling prevention and cleaning of nanofiltration membranes on page 219 etc. Chemical cleaning agents and processes, including alkaline cleaning and acid cleaning, are described on pages 220-221. Nitric acid, citric acid, phosphonic acid and phosphoric acid are mentioned as examples of acidic cleaning agents.

Various conditioning and cleaning methods for nanofiltration membranes (Desal-5 DK, Desal-5 DL and NF270 membranes) in the recovery of xylose by nanofiltration have been disclosed by E. Sjöman et al. in “Xylose recovery by nanofiltration from different hemicellulose hydrolyzate feeds”, Journal of Membrane Science 310 (2008), pages 268-277. In accordance with this document, the virgin membranes are conditioned with an alkaline cleaning agent (0.5% P3-Ultrasil-110) at 2 bar and 45° C. for 30 minutes and rinsed with ion free water, followed by nanofiltration of a first batch and a second batch of the hemicellulose hydrolyzate, from which xylose is to be separated. After each batch, the membranes are cleaned with an acidic and alkaline cleaning agent. The acidic cleaning is done with 5% acetic acid for 30 minutes at 50° C. at 2 bar. The alkaline cleaning is done with 1% P3-Ultrasil-110 for 10 minutes at 50° C. at 2 bar, followed by further 2 minutes after a stop of 30 minutes. Furthermore, the cleaning comprises rinsing with ion free water. It is recited that the cleaning is done to stabilize the membranes to long-term filtration-cleaning cycles. The cleaning methods described in this document have been carried out under relatively mild conditions, for example for relatively short periods of time and their purpose has been mostly to remove the fouling layer collected on the membrane during the nanofiltration of xylose solutions.

WO 02/053781 A1 and WO 02/053783 A1 mention the treatment of nanofiltration membranes with alkaline detergents and/or ethanol in the recovery of different chemical compounds, for example monosaccharides, such as xylose, by nanofiltration from a biomass hydrolysate. Furthermore, WO 2007/048879 A1 mentions the washing of nanofiltration membranes with an acidic washing agent in the recovery of xylose by nanofiltration from plant-based biomass hydrolysates.

Weng et al. discuss the retention of xylose and acetic acid at various initial acetic acid concentrations in “Separation of acetic acid from xylose by nanofiltration”, Separation and Purification Technology 67 (2009) 95-102. A negative retention of acetic acid was observed in the presence of xylose.

U.S. Pat. No. 5,279,739 discloses a polymeric composition useful in membrane technology such as nanofiltration. Suitable polymers for the composition include polyether sulfone, polysulfone and polyarylether sulfone. According to the examples, a suitable pore former may be added to the polymer composition prior to casting and hardening of the membranes. As suitable pore formers are mentioned low molecular weight organic compounds, inorganic salts and organic polymers. Furthermore, it is recited that other suitable pore formers include for example low molecular weight organic acids, such as acetic acid and propionic acid.

WO 2005/123157 A1 discloses a method of activating membranes useful in separation processes, such as nanofiltration and reverse osmosis methods, especially waste water treatment methods. In this method, the membrane is contacted for at least one day with a liquid activating agent comprising at least one acid and at least one surfactant. The acids may be selected from inorganic acids, organic acids and mixtures thereof. The organic acids may be selected from citric acid, adipic acid, succinic acid, glutaric acid, lactic acid, and maleic acid, for example. The surfactant may be selected from anionic surfactants, cationic surfactants, non-ionic surfactants, amphoteric surfactants and mixtures thereof. A treatment temperature of 25° C. is disclosed. It is recited that the method results in an improved permeate flux. It is also recited that the method results in decreased fouling of the membrane. This means better long term capacity, but not higher initial capacity. Furthermore, improvement of the flux of low molecular weight compounds (such as sugars) into the permeate is not disclosed or suggested.

Verissimo, S. et al disclose that the performance of reverse osmosis membranes, specifically composite hollow fiber membranes, can be improved by formic acid treatment in “Thin film composite hollow fiber membranes: An Optimized manufacturing method”, J. Membr. Sci. 264, (2005), 48-55. It appears from the document that the improved performance of the membranes refers to improved water permeability with NaCl rejections higher than 95%. In the same way as above, improvement of the flux of low molecular weight compounds other than water into the permeate is not disclosed or suggested.

U.S. Pat. No. 5,755,964 discloses a method of increasing the flux of a composite membrane having a polyamide layer by contacting the polyamide layer with an amine, such as ammonia. It is recited that the method makes it possible to control both the rejection rate and the flux of the membrane. The rejection rate is defined as the percentage of a particular dissolved material which does not flow through the membrane with solvent. The flux is defined as the flow rate at which solutions pass through the membrane. Consequently, the document does not disclose or suggest improvement of the flow (flux) of any particular dissolved material into the permeate.

One of the problems associated with known nanofiltration processes comprising post-treatment, conditioning and cleaning methods under relatively mild conditions as described above is that the initial throughput capacity of the membranes has not been sufficient and/or has not remained stabile in the long run, but decreases too quickly in successive nanofiltration runs. Consequently, there is a need for more efficient treatment methods to achieve increased membrane throughput capacity, without having a negative effect on the membrane structure and on the separation efficiency.

### DEFINITIONS RELATING TO THE INVENTION

“Membrane throughput capacity” is expressed as the flux of the compound to be separated, e.g. as xylose flux for the case where xylose is the target compound to be separated by the nanofiltration process.

“Flux” or “permeate flux” refers to the amount (liters or kg) of the solution that permeates through the nanofiltration membrane during one hour calculated per one square meter of the membrane surface, l/(m²h) or kg/(m²h).

“Water flux” refers to the amount (liters or kg) of water that permeates through the nanofiltration membrane during one hour calculated per one square meter of the membrane surface, l/(m²h) or kg/(m²h).

“Xylose flux” refers to the amount of xylose (g) that permeates through the nanofiltration membrane during one hour calculated per one square meter of the membrane surface, g/(m²h). Xylose flux may be determined by measuring the liquid flux and the content of dry substance and xylose in the permeate. The same definition applies to other target compounds to be separated. Consequently, for example “glucose flux” and “NaCl flux” are defined in the same way.

“Xylose purity” refers to the percentage (%) content of xylose in the dry substance of the permeate. The same definition applies to other target compounds to be separated. Consequently, for example “glucose purity” is defined in the same way.

“Separation efficiency” refers to the ability of the membranes in a nanofiltration process to separate the target compound(s) from the other compound in nanofiltration feed, expressed as the purity of the compound (% on DS) in the nanofiltration permeate compared to purity of the compound in the feed. The separation efficiency may also be expressed as the relation of two compounds to be separated from each other (their relation in the permeate compared to that in the feed).

“DS” refers to the dry substance content measured by Karl Fischer titration or by refractometry (RI), expressed as % by weight.

“MgSO₄ retention” refers to the observed retention of MgSO₄, which is a measure of the membrane selectivity toward MgSO₄ as shown below:

R_{MgSO4}=1−cₚ(MgSO₄)/c_{f}(MgSO₄)

where R_{MgSO4} is the observed retention of MgSO₄
cₚ(MgSO₄) is the concentration of MgSO₄ in the permeate (g/100 g solution)
c_{f}(MgSO₄) is the concentration of MgSO₄ in the feed (g/100 g solution).

“NaCl retention” refers to the observed retention of NaCl, defined in the same way as MgSO₄ retention above.

“Membrane treatment” refers to modifying a nanofiltration membrane with chemicals to increase the membrane throughput capacity. The membrane treatment in accordance with the invention may be performed by membrane manufacturers as post-treatment in the finishing stage of membrane manufacturing. The membrane treatment in accordance with the present invention may also be made as pretreatment in the nanofiltration operation.

“Membrane cleaning” and “membrane washing” refer to removing membrane preserving compounds from virgin membranes or removing foulants/contaminants/impurities which have been accumulated on the nanofiltration membranes (surfaces and pores thereof) during the nanofiltration operation or during storage of the nanofiltration membranes.

### DESCRIPTION OF THE INVENTION

An object of the present invention is thus to provide a process of treating nanofiltration membranes so as to alleviate the above-mentioned disadvantages relating non-sufficient or reduced membrane throughput capacity in known nanofiltration methods.

The invention relates to a process of treating polymeric nanofiltration membranes before separation of low molecular weight compounds from a solution containing the same by nanofiltration, wherein the treatment of the nanofiltration membranes is performed with a treatment liquid under conditions which enhance the flux of the low molecular weight compounds to the nanofiltration permeate while improving or essentially retaining the separation efficiency of the low molecular weight compounds.

In an embodiment of the invention, the treatment liquid is a solution comprising one or more compounds selected from organic acids and alcohols, organic sulfonic acids or sulfonates, and surfactants.

In an embodiment of the invention, the treatment liquid contains one or more of organic acids, one or more of acidic organic sulfonic acids or sulfonates and one or more of anionic tensides.

The organic acids may be selected from formic acid, acetic acid, propionic acid, lactic acid, oxalic acid, citric acid, itaconic acid, glycolic acid and aldonic acids. The aldonic acids may be selected from xylonic acid and gluconic acid, for example.

The alcohol may be selected from methanol, ethanol, n-propanol, isopropanol and glycerol, for example.

The organic sulfonic acids may be selected from alkyl aryl sulfonic acids and sulfonates, taurine, perfluorooctane sulfonic acid and Nafion (a sulfonated tetrafluoroethylene based fluoropolymer-copolymer).

The alkyl aryl sulfonic acids and sulfonates may be selected from toluene sulfonic acid and sodium dodecylbenzenesulfonate, for example.

The surfactants may be selected from anionic tensides and cationic tensides, for example.

In a typical embodiment of the invention, the treatment liquids are aqueous solutions containing one or more compounds recited above.

The concentration of the organic acids and alcohols in the treatment liquid may be 0.5% to 60% by weight, preferably 0.5% to 20% by weight, more preferably 0.5% to 10% by weight. The concentration of the sulfonic acids and sulfonates in the treatment liquid may be in the range of 0.1 to 10%, preferably 0.1 to 5% and more preferably 0.1 to 2% by weight. The concentration of the surfactants in the treatment liquid may be in the range of 0.01 to 10%, preferably 0.01 to 5% and more preferably 0.01 to 2% by weight.

In an embodiment of the invention, the treatment liquid is an aqueous liquid containing one or more organic acids, one or more of organic sulfonic acids and one or more of anionic tensides. In one specific embodiment of the invention, the organic acids are selected from a combination of citric acid and lactic acid, and the organic sulfonic acid is selected from alkyl aryl sulfonic acids.

In a further embodiment of the invention, the treatment liquid contains one or more of weak bases, preferably weak inorganic bases. The weak inorganic bases may be selected from weakly basic hydroxides, such as ammonium hydroxide, calcium hydroxide and magnesium hydroxide; weakly basic carbonates, such as sodium carbonate; and weakly basic oxides, such as calcium oxide and magnesium oxide.

The weak bases useful in the present invention may also selected from weak organic bases. The weak organic bases may be selected from acetone, pyridine, imidazole, benzimidazole; organic amines, such as alkyl amines, for example methyl amine; amino acids, such as histidine and alanine; phosphazene bases; and hydroxides of organic cations.

The weak bases useful in the present invention may also be selected from Lewis-bases, such as triethylamine, quinuclidine, acetonitrile, diethyllether, THF, acetone, ethyl acetate, diethylacetamide, dimethylsulfoxide, tetrahydrothiophene, and trimethyl phosphate.

The concentration of the weak bases in the treatment liquid may be 0.5% to 60% by weight, preferably 0.5% to 20% by weight, more preferably 0.5% to 10% by weight.

The weak bases recited above may be used alone or in combination with any of the organic acids and alcohols, organic sulfonic acids and sulfonates and surfactants recited above.

Furthermore, the treatment liquids may also be for example industrial process streams, which contain one or more of the recited compounds in concentrations mentioned above. The industrial process streams may be selected from various side streams from industrial plants, for example. Examples of useful industrial process streams are for instance side streams from wood processing industry and biorefineries, which may typically contain recited compounds in appropriate ranges. If appropriate, the industrial process streams may be diluted or concentrated to the desired concentration.

In specific embodiments of the invention, for example the following products may be used to provide the required treatment liquid: P3-Ultrasil 73, P3-Ultrasil 78, P3-Ultrasil 67 and P3-Ultrasil 53 (manufacturer Ecolab), Divosan Uniforce VS44, DIVOS 80-2 VM1, DIVOSAN PLUS VT53, Divos 80-6 VM35 and Divosan OSA-N VS37 (manufacturer Johnson Diversey), TriClean 211 and TriClean 217 (manufacturer Trisep), KLEEN MCT 103, KLEEN MCT403 and KLEEN MCT442 (manufacturer GE Water and Processes). The products may be used for example in dosages of 0.5 to 1% by volume as aqueous solutions.

As an example, P3-Ultrasil 73 contains following components (expessed in % by weight):

citric acid in an amount of 10 to 20%,

lactic acid in an amount of 5 to 10%,

an alkyl aryl sulfonic acid in an amount of 2 to 5%,

anionic tensides in an amount of less than 5%.

The treatment conditions (temperature and time) may vary within a wide range depending on the selected treatment liquid and the concentration thereof and the selected membrane, for example.

The treatment in accordance with the present invention may be performed at a temperature of 20° to 100° C., preferably 20° C. to 90° C., more preferably 30° C. to 85° C., still more preferably 45° C. to 80° C. and especially 55 to 80° C. In one embodiment of the invention, the treatment with weak bases is performed at a temperature of 20 to 40° C.

The treatment time may be 0.5 to 150 hours, preferably 1 to 100 hours, more preferably 1 to 70 hours.

In one embodiment of the invention, the treatment may comprise two or more successive steps with different treatment liquids, for example at least one step with a treatment liquid containing one or more alcohols, such as isopropanol, and at least one step with a treatment liquid containing one or more organic acids, such as acetic acid, in any desired sequence.

In a further embodiment of the invention, the treatment may comprise at least one step with a treatment liquid containing one or more weak inorganic bases and at least one step with a treatment liquid containing one or more organic acids, in any desired sequence. The weak inorganic base may be ammonium hydroxide and the organic acid may be lactic acid, for example.

In practice, the treatment may be performed by immersing, soaking or incubating the membrane elements in the treatment liquid. Mixing may be applied, if desired. The treatment may also be performed by recycling the pretreatment liquid in a nanofiltration apparatus provided with the membrane elements to be treated.

The treatment process of the present invention is followed by the actual nanofiltration for separating target compounds from various nanofiltration feeds.

Consequently, in a further embodiment of the invention, the process further comprises nanofiltration of a nanofiltration feed comprising low molecular weight compounds to obtain a nanofiltration retentate and a nanofiltration permeate, whereby said low molecular weight compound(s) are separated into the nanofiltration permeate with improved flux of the compound(s), while essentially retaining the separation efficiency. The nanofiltration is performed with nanofiltration membranes treated as described above. The flux improvement of the compound(s) is more than 20%, preferably more than 50%, more preferably more than 100% compared to the flux with untreated membranes.

The treatment of the present invention may be applied for example to the nanofiltration processes disclosed in WO 02/053781 A1 and 02/053783 A1 and WO 2007/048879 A1, which are incorporated herein by reference.

The compounds to be separated by the nanofiltration are typically low molecular weight compounds which have a molar mass of up to 360 g/mol.

The low molecular weight compounds to be separated may be selected from sugars, sugar alcohols, inositols, betaine, glycerol, amino acids, uronic acids, carboxylic acids, aldonic acids and inorganic and organic salts.

In one embodiment of the invention, the sugars are monosaccharides. The monosaccharides may be selected from pentoses and hexoses. The pentoses may be selected from xylose and arabinose. In one embodiment of the invention, the pentose is xylose.

The hexoses may be selected from glucose, galactose, rhamnose, mannose, fructose and tagatose. In one embodiment of the invention, the hexose is glucose.

The sugar alcohols may be selected from xylitol, sorbitol and erythritol, for example.

The carboxylic acids may be selected from citric acid, lactic acid, gluconic acid, xylonic acid and glucuronic acid.

The inorganic salts to be separated may be selected from monovalent salts, such as NaCl, NaHSO₄ and NaH₂PO₄ (monovalent anions, such as Cl⁻, HSO₄⁻ and H₂PO₄⁻), for example.

In a preferred embodiment of the invention, the compounds to be separated into the nanofiltration permeate may be product compounds, such as xylose, glucose and betaine.

In a further embodiment of the invention, the compounds to be separated into the nanofiltration permeate may be impurities, such as inorganic salts, especially monovalent salts like NaCl, NaHSO₄ and NaH₂PO₄. The compounds to be separated (from the impurities) into the nanofiltration retentate (concentrate) may comprise lactose, xylobiose and maltotriose, for example.

The starting material used as the nanofiltration feed in accordance with the present invention may be selected from plant-based biomass hydrolysates and biomass extracts and fermentation products thereof.

In one embodiment of the invention, the plant-based biomass hydrolysates may be derived from wood material from various wood species, such as hardwood, various parts of grain, bagasse, cocoanut shells, cottonseed skins etc. In one embodiment of the invention, the starting material may be a spent liquor obtained from a pulping process, for example a spent sulphite pulping liquor obtained from hardwood sulphite pulping. In a further embodiment of the invention, the starting material is a sugar beet based solution a or sugar cane based solution, such as molasses or vinasse.

In a further embodiment of the invention, the nanofiltration feed is selected from starch hydrolysates, oligosaccharide-containing surups, glucose syrups, fructose syrups, maltose syrups and corn syrups.

In a further embodiment of the invention, the nanofiltration feed may be a lactose-containing dairy product, such as whey.

In one embodiment of the invention, the nanofiltration comprises the separation of xylose from a spent liquor obtained from a pulping process, for example a spent sulphite pulping liquor obtained from hardwood sulphite pulping. Xylose is recovered as a product from the nanofiltration permeate.

In a further embodiment of the invention, the nanofiltration comprises the separation of betaine from a sugar beet based solution, such as molasses or vinasse. Betaine may be recovered as a product from the nanofiltration permeate.

In a still further embodiment of the invention, the nanofiltration comprises the separation of glucose from a glucose syrup, such as dextrose corn syrup. Glucose is recovered as a product from the nanofiltration permeate.

In a still further embodiment of the invention, the nanofiltration comprises the separation of inorganic salts, especially monovalent salts, from a lactose-containing dairy product, for example whey. The salts are separated as impurities into the nanofiltration permeate.

The polymeric nanofiltration membranes useful in the present invention include, for example, aromatic polyamide membranes such as polypiperazineamide membranes, aromatic polyamine membranes, polyether sulfone membranes, sulfonated polyether sulfone membranes, polyester membranes, polysulfone membranes, polyvinyl alcohol membranes and combinations thereof. Composite membranes composed of layers of one or more of the above-mentioned polymeric materials and/or other materials are also useful in the present invention.

Preferred nanofiltration membranes are selected from polyamide membranes, especially polypiperazineamide membranes. As examples of useful membranes can be mentioned Desal-5 DL, Desal-5 DK and Desal HL by General Electrics Osmonics Inc., NF 270, NF 245 and NF 90 by Dow Chemicals Co., NE40 and NE70 by Woongjin Chemicals Co, Alfa-Laval NF, AlfaLaval NF 10 and Alfa-Laval NF 20 by Alfa-Laval Inc and TriSep TS40 by TriSep Co and Hydranautics 84200 ESNA 3J by Nitto Denko Co.

The nanofiltration membranes useful for the treatment of the invention typically have a cut-off size of 150 to 1 000 g/mol, preferably 150 to 250 g/mol.

The nanofiltration membranes which are useful in the present invention may have a negative or positive charge. The membranes may be ionic membranes, i.e. they may contain cationic or anionic groups, but even neutral membranes are useful. The nanofiltration membranes may be selected from hydrophobic and hydrophilic membranes.

Typical forms of the membranes are spiral wound membranes and flat sheet membranes assembled in plate and frame modules. The membrane configuration may be also selected e.g. from tubes, and hollow fibers.

In one embodiment of the invention, the treatment is done on non-used virgin membranes, before the membranes are taken into use. In another embodiment of the invention, the treatment may be done on used membranes before a new nanofiltration. The treatment may be regularly repeated for example within intervals of 3 to 6 months during the nanofiltration use.

The nanofiltration conditions (such as the temperature and pressure, the dry substance content of the nanofiltration feed and the content of the low molecular weight compound in the nanofiltration feed) may vary depending on the selected starting material (nanofiltration feed), the compound to be separated and the selected membrane. The nanofiltration conditions may be selected for example from those described in WO 02/053781 A1 and 02/053783 A1 and WO 2007/048879 A1, which are incorporated herein by reference.

The nanofiltration temperature may be in the range of 5 to 95° C., preferably 30 to 80° C. The nanofiltration pressure may be in the range of 10 to 50 bar, typically 15 to 35 bar.

The dry substance content of the nanofiltration feed may be in the range of 5% to 60% by weight, preferably 10% to 40% by weight, more preferably 20% to 35% by weight.

The content of the low molecular weight compounds, e.g. xylose or betaine, in nanofiltration feeds selected from plant-based biomass hydrolysates and extracts may be in the range of 10 to 65% on DS, preferably 30 to 65% on DS. The content of the low molecular weight compounds, e.g. glucose, in nanofiltration feeds selected from starch hydrolysates, oligosaccharidecontaining surups, glucose syrups, fructose syrups, maltose syrups and corn syrups may be in the range of 90 to 99%, preferably 94 to 99%.

It was found that the preteatment process of the present invention provides a considerable increase in the membrane throughput capacity for the low molecular weight compounds which are separated into the nanofiltration permeate, while also improving the permeate flux. For example in the separation of xylose, the increase in the capacity may be even up to 300% or higher, measured for xylose separation as the increased xylose flux through the membrane, while retaining the separation efficiency. It was also found that the achieved capacity increase was stabile during repeated nanofiltration cycles. At the same time, the separation efficiency measured for example as the purity of xylose or as the separation of xylose from glucose remained the same or even improved along with the higher capacities.

In one embodiment of the invention, the flux of the low molecular weight compounds to the nanofiltration permeate is in the range of 10 to 20 000 g/m²h.

In the separation of sugars, the flux of the sugars to the nanofiltration permeate may be in the range of 20 to 15 000 g/m²h, preferably 100 to 8 000 g/m²h, most preferably 100 to 4 000 g/m²h.

In the separation of xylose, the flux of xylose to the nanofiltration permeate may be in the range of 100 to 15 000 g/m²h, preferably 300 to 15 000 g/m²h, most preferably 1 000 to 15 000 g/m²h.

In the separation of glucose, the flux of glucose to the nanofiltration permeate may be in the range of 200 to 15 000 g/m²h, preferably 200 to 10 000 g/m²h, most preferably 200 to 8 000 g/m²h.

In the separation of inorganic salts, the flux of the salts to the nanofiltration permeate may be in the range of 20 to 2000 g/m²/h, preferably 40 to 1500 g/m²/h and more preferably 80 to 1000 g/m²/h.

In one specific embodiment of the invention, the invention relates to a process of separating and recovering xylose from a xylose-containing solution by nanofiltration with a polymeric nanofiltration membrane, comprising

treating the membrane with an organic liquid comprising citric acid, lactic acid, an alkyl aryl sulfonic acid and anionic tensides in the following conditions:
concentration of citric acid 0.5 to 20% by weight,
concentration of lactic acid 0.5 to 20% by weight
concentration of the alkyl aryl sulfonic acid 0.1 to 10% by weight,
concentration of the anionic tensides 0.1 to 10% by weight,
treatment temperature 50 to 70° C., and
treatment time 2 to 70 hours,

to obtain a treated nanofiltration membrane, followed by

nanofiltering the xylose-containing solution with the treated nanofiltration membrane with a xylose flux of 100 to 15 000 g xylose/m²h to the nanofiltration permeate, and

recovering xylose from the nanofiltration permeate.

In a further specific embodiment of the invention, the invention relates to a process for separating and recovering xylose from a xylosecontaining solution by nanofiltration with a polymeric nanofiltration membrane, comprising in any desired sequence

a step of treating the membrane with a treatment liquid containing lactic acid in the following conditions:
concentration of lactic acid 20 to 60% by weight,
treatment temperature 50 to 70° C., and
treatment time 2 to 80 hours, and

a step of treating the membrane with a treatment liquid containing ammonium hydroxide in the following conditions:
concentration of ammonium hydroxide 0.1 to 10% by weight,
treatment temperature 20 to 40° C.,
treatment time 2 to 80 hours,

to obtain a treated nanofiltration membrane, followed by

nanofiltering the xylose-containing solution with the treated nanofiltration membrane with a xylose flux of 100 to 15 000 g xylose/m²h to the nanofiltration permeate, and

recovering xylose from the nanofiltration permeate.

### EXAMPLES

The invention will now be described in greater detail with following examples, which are not construed as limiting the scope of the invention.

The following membrane was used in the examples:
Desal-5 DK (manufacturer General Electrics (GE) Osmonics Inc.),
Desal-5 DL (manufacturer GE Osmonics Inc.),
NF 245 (manufacturer Dow Chemicals Co.),
Alfa-Laval NF, Alfa-Laval NF 10 and Alfa-Laval NF 20 (manufacturer Alfa-Laval Inc.),
Trisep TS40 (manufacturer TriSep Co.), and
Hydranautics 84200 ESNA 3J (manufacturer Nitto Denko Co).

HPLC (for the determination of xylose and glucose) refers to liquid chromatography. RI detection was used.

The tests with pure water represent reference tests (no pretreatment).

### Example 1

### Xylose Flux Test after Treatment of GE Osmonics Desal 5 DK Membrane with Various Compounds/Compositions

A membrane treatment test was carried out with flat sheets cut from spiral wound elements. The nanofiltration membrane tested was GE Osmonics Desal 5 DK membrane. The filtration unit used in the test was Alfa Laval LabStak M20.

All the tested membrane sheets were pre-washed with ion free water for 48 hours at 25° C. to remove all membrane preserving compounds. Then the membranes were washed with an alkaline washing agent for 30 minutes by soaking in 0.1% alkaline solution (Ecolab Ultrasil 112) at 30° C. The membranes were flushed with ion free water. The next step was to soak the membranes for 2 minutes in 0.1% acetic acid at 30° C. followed by flushing with IEX (ion exchanged) water.

After the pre-washing steps, the membrane sheets were treated by incubation in various test liquids at 70° C. for 24 to 72 hours. The test liquids were pure water, sodium dodecyl sulfate, metabisulphite, N-N-dimethylacetamide, formic acid, acetic acid, acidic washing agent (Ecolab P3-Ultrasil 73) with varying concentrations. After the soaking treatment, the membrane sheets were flushed well with ion free water before assembling them to the nanofiltration test unit.

A xylose flux test with the treated membranes was carried out with a 23% DS industrial xylose solution, obtained from chromatographically separated xylose fraction of Mg-based acid spent sulphite pulping liquor, obtained according to WO 021 053 783 A1. The xylose flux test was done at 30 bar/70° C. using 3 m/s cross flow velocity. The filtrations were done with a reflux mode, e.g. all permeates were introduced back into the feed tank. The filtration time before the measurements and sample taking was 30 minutes.

The permeate flux values were registered and the permeate samples were analysed with HPLC to measure the xylose content for the calculation of xylose flux. The membrane treatment methods, xylose fluxes, permeate fluxes, permeate DS and xylose purities in the permeate are presented in Table 1.

TABLE 1

| | Permeate | Xylose | Permeate |
|---|---|---|---|
| | flux | flux | xylose purity |
| Membrane treatment method | kg/m2/h | g/m2/h | % on DS |
| Pure water, 70° C., 72 h | 1.5 | 233 | 59.4 |
| Pure water, 70° C., 72 h | 1.5 | 222 | 59.3 |
| 40% Formic acid, 70° C., 72 h | 4.1 | 640 | 58.9 |
| 45% Acetic acid, 70° C., 84 h | 4.0 | 604 | 59.5 |
| 0.5% P3-Ultrasil 73, 72 h, 70° C. | 2.5 | 388 | 59.3 |
| 1% P3-Ultrasil 73, 72 h, 70° C. | 9.0 | 1374 | 59.2 |
| 10% P3-Ultrasil 73, 72 h, 70° C. | 71.0 | 13846 | 52.7 |
| 200 g/L Na-dodecyl sulfate, | 1.6 | 238 | 59.6 |
| 24 h, 50° C. | | | |
| 650 g/L Metabisulphite, 24 h, 50° C. | 1.7 | 257 | 58.5 |
| 19.6% N-N-dimethylacetamide, 24 h, | 1.6 | 247 | 58.7 |

### Example 2

### A Further Xylose Flux Test after Treatment of GE Osmonics Desal 5 DK Membrane with Various Compounds/Compositions

A membrane treatment test was carried out with flat sheets cut from spiral wound elements. The nanofiltration membrane tested was GE Osmonics Desal 5 DK membrane. The filtration unit used in the test was Alfa Laval LabStak M20.

After the pre-washing steps in accordance with Example 1, the membrane sheets were treated by incubation in various test liquids at 70° C. for 24 to 72 hours. The test liquids in this example were pure water, sodium dodecyl sulfate, Fennopol K3450 (cationic surfactant, manufactured by Kemira) hexane, chitosan, gluconic acid formic acid, acetic acid, acidic washing agent (Ecolab P3-Ultrasil 73) with varying concentrations. After the soaking treatment, the membrane sheets were flushed well with ion free water before assembling them to the nanofiltration test unit.

A xylose flux test with the treated membranes was carried out with a 23% DS industrial xylose solution in accordance with Example 1.

The permeate flux values were registered and the permeate samples were analysed with HPLC to measure the xylose content for the calculation of xylose flux. The membrane treatment methods, xylose fluxes, permeate fluxes, permeate DS and xylose purities in the permeate are presented in Table 2.

TABLE 2

| | Xylose | Xylose |
|---|---|---|
| | flux | purity |
| Membrane treatment method | g/m2/h | % on DS |
| IEX-water, 72 h, 70° C. | 241 | 59.8 |
| IEX-water, 72 h, 70° C. | 242 | 59.9 |
| 0.1% FENNOPOL K3450 24 h, 25° C. | 286 | 59.6 |
| 100% Hexane, 7 h, 25° C. | 288 | 60.2 |
| 0.1% Na-dodecyl sulfate, 24 h, 25° C. | 323 | 59.2 |
| 0.5% Chitosan, pH 4, 24 h, 25° C. | 307 | 60.4 |
| 40% Gluconic acid 72 h, 70° C. | 424 | 59.9 |
| 45% Acetic acid, 84 h, 70° C. | 689 | 59.8 |
| 40% Formic Acid, 72 h, 70° C. | 869 | 60.2 |
| 0.1% P3-Ultrasil 73 72 h, 70° C. | 311 | 60.0 |
| 1% P3-Ultrasil 73, 72 h, 70° C. | 1566 | 57.4 |

### Example 3

### A Further Xylose Flux Test after Treatment of GE Osmonics Desal 5 DK Membrane with Various Compounds/Compositions

A membrane treatment test was carried out with flat sheets cut from spiral wound elements. The nanofiltration membrane tested was GE Osmonics Desal 5 DK membrane. The filtration unit used in the test was Alfa Laval LabStak M20.

After the pre-washing steps in accordance with Example 1, the membrane sheets were treated by incubation in various test liquids at 70° C. for 24 to 72 hours. The test liquids in this example were pure water, sodium dodecyl sulfate (SDS), acetic acid, acidic washing agent (Ecolab P3-Ultrasil 73) with varying concentrations, incubation times and temperatures. After the soaking treatment, the membrane sheets were flushed well with ion free water before assembling them to the nanofiltration test unit.

A xylose flux test with the treated membranes was carried out with a 23% DS industrial xylose solution in accordance with Example 1.

The permeate flux values were registered and the permeate samples were analysed with HPLC to measure the xylose content for the calculation of xylose flux. The membrane treatment methods, xylose fluxes, permeate fluxes and xylose purities in the permeate are presented in Table 3.

TABLE 3

| | Permeate | Xylose | Xylose |
|---|---|---|---|
| | flux | flux | purity |
| Membrane treatment method | kg/m2/h | g/m2/h | % on DS |
| IEX water, 72 h | 1.2 | 167 | 59.7 |
| 200 g/L SDS, 24 h | 1.3 | 178 | 60.8 |
| IEX water, 72 h | 1.0 | 139 | 58.5 |
| 40% Formic acid, 72 h | 2.6 | 361 | 60.2 |
| 45% Acetic acid, 84 h | 2.5 | 353 | 61.0 |
| 0.5% P3-Ultrasil 73, 72 h, | 1.9 | 263 | 59.6 |
| 1% P3-Ultrasil 73, 72 h, | 5.3 | 736 | 60.0 |

### Example 4

### Xylose Flux Test after Treatment of GE Osmonics Desal 5 DL Membrane with P3-Ultrasil in Various Concentrations and Conditions

A membrane treatment test was carried out with flat sheets cut from spiral wound elements. The nanofiltration membrane tested was GE Osmonics Desal 5 DL membrane. The filtration unit used in the test was Alfa Laval LabStak M20.

After the pre-washing steps in accordance with Example 1, the membrane sheets were treated by incubation in various test liquids at 60 to 70° C. for 3 to 110 hours. The test liquids in this example were pure water and acidic washing agent (Ecolab P3-Ultrasil 73) with varying concentrations, incubation times and incubation temperatures. After the soaking treatment, the membrane sheets were flushed well with ion free water before assembling them to the nanofiltration test unit.

The first test with the pre-treated membranes was a MgSO₄ retention test. The MgSO₄ retention test was carried out with a 2000 ppm MgSO₄ solution at 8.3 bar/25° C., with a reflux mode, e.g. all permeates were introduced back into the feed tank. The filtration time before the measurements and sample taking and was 60 minutes.

A xylose flux test with the treated membranes was carried out with a 23% DS industrial xylose solution in accordance with Example 1.

The permeate flux values were registered and the permeate samples were analysed with HPLC to measure the xylose content for the calculation of xylose flux. The membrane treatment methods, xylose fluxes, MgSO₄ retentions and xylose purities in the permeate are presented in Table 4.

TABLE 4

| | Treatment | | | | | |
|---|---|---|---|---|---|---|
| | chemical | Treatment | | | Xylose | Xylose |
| Membrane | concentration | temperature | Treatment | Retention | flux | purity |
| treatment liquid | %-(w/w) | ° C. | time h | MgSO4 % | g/m2/h | % on DS |
| Water | 0 | 72 | 110 | 98.6 | 559 | 59.9 |
| P3-Ultrasil 73 | 2 | 65 | 8 | 99.3 | 657 | 60.4 |
| P3-Ultrasil 73 | 4 | 70 | 5 | 98.9 | 578 | 59.2 |
| P3-Ultrasil 73 | 4 | 70 | 11 | 97.6 | 855 | 59.7 |
| P3-Ultrasil 73 | 7 | 65 | 3 | 99.4 | 627 | 62 |
| P3-Ultrasil 73 | 7 | 65 | 13 | 99 | 854 | 60.6 |
| P3-Ultrasil 73 | 7 | 65 | 8 | 99.4 | 682 | 60.5 |
| P3-Ultrasil 73 | 7 | 65 | 8 | 99.5 | 682 | 61.8 |
| P3-Ultrasil 73 | 7 | 65 | 8 | 99.3 | 707 | 60.2 |
| P3-Ultrasil 73 | 10 | 65 | 8 | 99.3 | 680 | 60.6 |
| P3-Ultrasil 73 | 10 | 65 | 8 | 99.3 | 695 | 59.8 |
| P3-Ultrasil 73 | 10 | 65 | 8 | 99 | 768 | 60.6 |
| P3-Ultrasil 73 | 10 | 60 | 11 | 99.1 | 636 | 59.5 |
| P3-Ultrasil 73 | 10 | 70 | 11 | 95.5 | 1069 | 59.1 |
| P3-Ultrasil 73 | 10 | 70 | 5 | 98.9 | 631 | 59.4 |
| P3-Ultrasil 73 | 12 | 65 | 8 | 99.5 | 628 | 61.5 |

### Example 5

### Xylose and Glucose Flux Test after Treatment of Various Membranes with Various Compounds/Compositions

A membrane treatment test was carried out with flat sheets cut from spiral wound elements. The nanofiltration membrane tested was GE Osmonics Desal 5 DK) membrane, and Dow NF245 membrane. The filtration unit used in the test was Alfa Laval LabStak M20.

After the pre-washing steps in accordance with Example 1, the membrane sheets were treated by incubation in various test liquids at 70° C. for 3 to 7 hours. The test liquids in this example were pure water, formic acid and acidic washing agent (Ecolab P3-Ultrasil 73) with varying concentrations. After the soaking treatment, the membrane sheets were flushed well with ion free water before assembling them to the nanofiltration test unit.

A xylose flux test with the treated membranes was carried out with a 23% DS industrial xylose solution in accordance with Example 1. Furthermore, a glucose flux test was performed in an equivalent way.

The permeate flux values were registered and the permeate samples were analysed with HPLC to measure the xylose and glucose content for the calculation of xylose and glucose flux. The membrane treatment methods, xylose fluxes and xylose purities in the permeate as well as glucose fluxes and glucose purities in the permeate measured with respective membranes are presented in Table 5.

TABLE 5

| | Glucose | Glucose | Xylose | Xylose |
|---|---|---|---|---|
| Membrane treatment method | purity | flux | flux | purity |
| membrane, chemical, time h | % on ds | g/m2/h | g/m2/h | % on DS |
| DK, Water, 7 h | 2.6 | 8.7 | 185.0 | 55.8 |
| DK, 1% P3-Ultrasil 73, 1 h | 2.9 | 14.9 | 292.0 | 56.5 |
| DK, 1% P3-Ultrasil 73, 7 h | 2.7 | 13.3 | 279.0 | 55.8 |
| DK, 2% P3-Ultrasil 73, 1 h | 2.8 | 14.9 | 299.0 | 55.4 |
| DK, 2% P3-Ultrasil 73, 7 h | 2.7 | 15.1 | 304.0 | 55.1 |
| DK, 40% Formic acid, 7 h | 2.9 | 23.4 | 460.0 | 56.4 |
| Dow NF245, Water, 7 h | 2.9 | 21.3 | 414.0 | 56.0 |
| Dow NF245, 7% | 3.0 | 26.7 | 496.0 | 56.2 |
| P3-Ultrasil 73, 3 h | | | | |
| Dow NF245, 7% | 3.2 | 30.8 | 557.0 | 57.4 |
| P3-Ultrasil 73, 7 h | | | | |

### Example 6

### Xylose Flux Test after Treatment of Dow NF245 Membrane with P3-Ultrasil 73

A membrane treatment test was carried out with flat sheets. The nanofiltration membrane tested was Dow NF 245 membrane. The filtration unit used in the test was Alfa Laval LabStak M20.

After the pre-washing steps in accordance with Example 1 (the acetic acid soaking at 25° C. instead of 30° C.), the membrane sheets were treated by incubation in various test liquids at 68° C. for 24 to 72 hours. The test liquids were pure water and acidic washing agent (Ecolab P3-Ultrasil 73) with varying concentrations. After the soaking treatment, the membrane sheets were flushed well with ion free water before assembling them to the nanofiltration test unit.

A xylose flux test with the treated membranes was carried out with a 23% DS industrial xylose solution in accordance with Example 1.

The permeate flux values were registered and the permeate samples were analysed with a conductivity meter and HPLC to measure the salt content and the xylose content for the calculation of salt retention and xylose flux. The membrane treatment methods, xylose fluxes and salt retentions are presented in Table 6.

TABLE 6

| | | | | Salt retention, |
|---|---|---|---|---|
| | Conc., | Treatment | Xylose flux, | % of feed |
| Treatment liquid | weight-% | time, h | g/m2/h | conductivity |
| Pure H2O | | 7 | 684 | 72% |
| P3-Ultrasil 73 | 1.0% | 16 | 688 | 67% |
| P3-Ultrasil 73 | 1.0% | 24 | 4379 | 61% |

### Example 7

### Xylose Flux Test after Treatment of Alfa-Laval NF Membranes with Lactic Acid

A membrane treatment test was carried out with flat sheets. The nanofiltration membrane tested were three Alfa-Laval NF membranes named NF, NF 10 and NF 20. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 68° C. for 7 to 72 hours. The test liquids were pure water and lactic acid with varying concentrations. After the soaking treatment, the membrane sheets were flushed well with ion free water before assembling them to the nanofiltration test unit.

A xylose flux test with the treated membranes was carried out with a 23% DS industrial xylose solution in accordance with Example 1.

The permeate flux values were registered and the permeate samples were analysed with a conductivity meter and HPLC to measure the salt content and the xylose content for the calculation of salt retention and xylose flux. The membrane treatment methods, xylose fluxes, xylose purities in the permeate as well as the salt retentions measured with respective membranes are presented in Table 7.

TABLE 7

| | | | | Xylose | Salt retention, | Xylose |
|---|---|---|---|---|---|---|
| | Treatment | Conc.. | | flux, | % of feed | purity |
| Membrane | liquid | weight-% | Time, h | g/m2/h | conductivity | % on DS |
| Alfa-Laval NF | IEX water | | 7 | 537 | 81% | 59.3 |
| Alfa-Laval NF | Lactic acid | 40% | 72 | 897 | 79% | 55.1 |
| Alfa-Laval NF | Lactic acid | 60% | 72 | 956 | 81% | 55.8 |
| Alfa-Laval NF 10 | IEX water | | 7 | 306 | 76% | 59.0 |
| Alfa-Laval NF 10 | Lactic acid | 40% | 72 | 798 | 82% | 56.3 |
| Alfa-Laval NF 10 | Lactic acid | 60% | 72 | 847 | 82% | 56.0 |
| Alfa-Laval NF 20 | IEX water | | 7 | 588 | 72% | 55.6 |
| Alfa-Laval NF 20 | Lactic acid | 40% | 72 | 1215 | 80% | 57.6 |
| Alfa-Laval NF 20 | Lactic acid | 60% | 72 | 1342 | 81% | 55.9 |

### Example 8

### Xylose Flux Test after Treatment of TriSep TS40 and Osmonics Desal 5 DL Membranes with Lactic Acid

A membrane treatment test was carried out with flat sheets. The nanofiltration membranes tested were TriSep TS40 and GE Osmonics Desal 5 DL. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 68° C. for 7 to 72 hours. The test liquids were pure water and lactic acid with varying concentrations. After the soaking treatment, the membrane sheets were flushed well with ion free water before assembling them to the nanofiltration test unit.

The first test with the pre-treated membranes was a MgSO₄ retention test. The test was carried out with a 2000 ppm MgSO₄ solution at 8.3 bar/25° C., with a reflux mode, e.g. all permeates were introduced back into the feed tank. The filtration time before the measurements and sample taking and was 60 minutes.

The second test with the pre-treated membranes was a NaCl retention flux test. The test was carried out with a 5000 ppm NaCl solution at 8.3 bar/25° C., with a reflux mode, e.g. all permeates were introduced back into the feed tank. The filtration time before the measurements and sample taking and was 60 minutes.

A xylose flux test with the treated membranes was carried out with a 23% DS industrial xylose in accordance with Example 1.

The permeate flux values were registered and the permeate samples were analysed with HPLC to measure the xylose content for the calculation of xylose flux. The membrane treatment methods and the results of MgSO4, NaCl and xylose tests with respective membranes are presented in Table 8.

TABLE 8

| | | | | | NaCl | | Xylose |
|---|---|---|---|---|---|---|---|
| | Treatment | Conc., | Treatment | NaCl | flux, | MgSO4 | flux, |
| Membrane | liquid | weight-% | time, h | retention, % | g/m2/h | retention, % | g/m2/h |
| Desal 5 DL | IEX water | 0 | 24 | 18.0 | 167 | 97.8 | 508 |
| Desal 5 DL | Lactic acid | 40 | 24 | 14.3 | 270 | 97.7 | 940 |
| Trisep TS40 | IEX water | 0 | 24 | 26.3 | 211 | 98.7 | 309 |
| Trisep TS40 | Lactic acid | 20 | 24 | 24.2 | 278 | 99.3 | 446 |
| Trisep TS40 | Lactic acid | 40 | 24 | 23.9 | 306 | 98.8 | 479 |
| Trisep TS40 | Lactic acid | 60 | 24 | 24.0 | 333 | 99.1 | 496 |
| Trisep TS40 | IEX water | 0 | 24 | 27.3 | 240 | 99.2 | 328 |

### Example 9

### Xylose Flux Test after Treating Hydranautics 84200 ESNA 3J NF Membrane with Lactic Acid

A membrane treatment test was carried out with flat sheets. The nanofiltration membrane tested was Hydranautics 84200 ESNA 3J. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 68° C. for 7 to 72 hours. The test liquids were pure water and 40% lactic acid. After the soaking treatment, the membrane sheets were flushed well with ion free water before assembling them to the nanofiltration test unit. A xylose flux test with the treated membranes was carried out with a 23% DS industrial xylose solution in accordance with Example 1.

The permeate flux values were registered and the permeate samples were analysed with a conductivity meter and HPLC to measure the salt content and the xylose content for the calculation of salt retention and xylose flux. The membrane treatment methods, xylose fluxes and xylose purities in the permeate as well as salt retentions are presented in Table 9.

TABLE 9

| | | | | | Salt | Xylose |
|---|---|---|---|---|---|---|
| | | | | Xylose | retention, | purity, |
| | Treatment | Conc., | | flux, | % of feed | % on |
| Membrane | liquid | weight-% | Time, h | g/m2/h | conductivity | DS |
| Hydranautics 84200 ESNA 3J | IEX water | 0 | 7 | 233 | 56% | 52.2 |
| Hydranautics 84200 ESNA 3J | Lactic acid | 40 | 72 | 537 | 60% | 52.3 |

### Example 10

### Xylose Flux Test after Treatment of GE Osmonics Desal 5 DL Membrane with Various Compounds/Compositions

A membrane treatment test was carried out with flat sheets. The nanofiltration membrane tested was GE Osmonics Desal 5 DL membrane. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 68° C. for 24 to 72 hours. The test liquids were pure water, acidic washing agent (Ecolab P3-Ultrasil 73) and sodium dodecylbenzenesulfonate with varying concentrations. After the soaking treatment, the membrane sheets were flushed well with ion free water before assembling them to the nanofiltration test unit.

A xylose flux test with the treated membranes was carried out with a 23% DS industrial xylose solution in accordance with Example 1.

The permeate flux values were registered and the permeate samples were analysed with a conductivity meter and with HPLC to measure salt and the xylose content for the calculation of salt retention and the xylose flux. The membrane treatments, the xylose fluxes and salt retentions are presented in Table 10.

TABLE 10

| | | | | | Salt |
|---|---|---|---|---|---|
| | | Conc., | | Xylose | retention, |
| | | weight- | Time, | flux, | % of feed |
| Membrane | Treatment liquid | % | h | g/m2/h | conductivity |
| Desal 5 DL | IEX water | 0 | 7 | 417 | 61 |
| Desal 5 DL | P3-Ultrasil 73 | 1 | 16 | 528 | 68 |
| Desal 5 DL | P3-Ultrasil 73 | 1 | 24 | 438 | 60 |
| Desal 5 DL | P3-Ultrasil 73 | 1 | 32 | 530 | 68 |
| Desal 5 DL | P3-Ultrasil 73 | 1 | 48 | 679 | 68 |
| Desal 5 DL | Sodium dodecyl- | 0.5 | 24 | 475 | 62 |
| | benzenesulfonate | | | | |
| Desal 5 DL | Sodium dodecyl- | 0.5 | 48 | 770 | 67 |
| | benzenesulfonate | | | | |

### Example 11

### Xylose Flux Test after Treatment of GE Osmonics Desal 5 DL Membrane with Ammonium Hydroxide

A membrane treatment test was carried out with flat sheets. The nanofiltration membrane tested was GE Osmonics Desal 5 DL membrane. The filtration unit used in the test was Alfa Laval LabStak M2.

After the pre-washing steps in accordance with Example 1 (acetic acid soaking at 25° C. instead of 30° C.), the membrane sheets were treated by incubation in various test liquids at 25° C. for 24 to 72 hours. The test liquids were pure water and ammonium hydroxide with varying concentrations. After the soaking treatment, the membrane sheets were flushed well with ion free water before assembling them to the nanofiltration test unit.

A xylose flux test with the treated membranes was carried out in a similar manner as in Example 1.

The permeate flux values were registered and the permeate samples were analysed with a conductivity meter and with HPLC to measure the salt content and the xylose content for the calculation of salt retention and the xylose flux. The membrane treatments, xylose fluxes and salt retentions measured with respective membranes are presented in Table 11.

TABLE 11

| | | | | Xylose | Salt retention, |
|---|---|---|---|---|---|
| | Treatment | Conc., | Time, | flux, | % of feed |
| Membrane | liquid | weight-% | h | g/m2/h | conductivity |
| Desal 5 DL | IEX water | 0 | 72 | 419 | 73 |
| Desal 5 DL | Ammonium | 1 | 72 | 686 | 67 |
| | hydroxide | | | | |
| Desal 5 DL | Ammonium | 5 | 24 | 503 | 72 |
| | hydroxide | | | | |
| Desal 5 DL | Ammonium | 5 | 72 | 750 | 63 |
| | hydroxide | | | | |

### Example 12

### Xylose Flux Test after Treatment of GE Osmonics Desal 5 DL Membrane with Ammonium Hydroxide and Lactic Acid in Two Steps

A membrane treatment test was carried out with flat sheets. The nanofiltration membrane tested was GE Osmonics Desal 5 DL membrane. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 25° C. or 68° for 24 or 72 hours, followed by an optional second incubation according to Table 12. The test liquids were pure water, 40% lactic acid and 5% ammonium hydroxide. After the soaking treatment, the membrane sheets were flushed well with ion free water before assembling them to the nanofiltration test unit.

A xylose flux test with the treated membranes was carried out in a similar manner as in Example 1.

The permeate flux values were registered and the permeate samples were analysed with a conductivity meter and with HPLC to measure the salt content and the xylose content for the calculation of salt retention and the xylose flux. The membrane treatment methods, xylose fluxes and salt retentions measured with respective membranes are presented in Table 12.

TABLE 12

| | | | Salt |
|---|---|---|---|
| | Membrane | Xylose | retention. |
| Membrane | treatment | flux, | % of feed |
| treatment method 1 | method 2 | g/m2/h | conductivity |
| IEX water (1) | — | 582 | 60 |
| 5% Ammonium hydroxide, | — | 531 | 71 |
| 25° C., 72 h | | | |
| 40% Lactic acid, 68° C., 24 h | — | 1033 | 77 |
| 40% Lactic acid, 68° C., 40 h | 5% Ammonium | 1324 | 76 |
| | hydroxide | | |
| | 25° C., 24 h | | |
| 40% Lactic acid, 68° C., 48 h | 5% Ammonium | 1198 | 64 |
| | hydroxide, | | |
| | 25° C., 24 h | | |
| 40% Lactic acid, 68° C., 72 h | 5% Ammonium | 1340 | 76 |
| | hydroxide, | | |
| | 25° C., 24 h | | |
| 5% Ammonium hydroxide, | 40% Lactic acid, | 1077 | 71 |
| 25° C., 24 h | 68° C., 24 h | | |
| 5% Ammonium hydroxide, | 40% Lactic acid, | 1039 | 75 |
| 25° C., 48 h | 68° C., 24 h | | |
| 5% Ammonium hydroxide, | 40% Lactic acid, | 1235 | 75 |
| 25° C., 72 h | 68° C., 24 h | | |

### Example 13

### Xylose Flux Test after Treatment of TriSep TS40 NF Membrane, with Ammonium Hydroxide

A membrane treatment test was carried out with flat sheets. The nanofiltration membrane tested was TriSep TS40 membrane. The filtration unit used in the test was Alfa Laval LabStak M20.

After the pre-washing steps in accordance with example 1 (acetic acid soaking at 25° C. instead of 30° C.), the membrane sheets were treated by incubation in various test liquids at 25° C. for 24 to 72 hours. The test liquids were pure water and ammonium hydroxide with varying concentrations. After the soaking treatment, the membrane sheets were flushed well with ion free water before assembling them to the nanofiltration test unit.

A xylose flux test with the treated membranes was carried out in a similar manner as in Example 1.

The permeate flux values were registered and the permeate samples were analysed with a conductivity meter and with HPLC to measure the salt content and the xylose content for the calculation of salt retention and the xylose flux. The membrane treatment methods, the xylose fluxes and salt retentions measured with respective membranes are presented in Table 13.

TABLE 13

| | | | | Xylose | Salt retention, |
|---|---|---|---|---|---|
| | Treatment | Conc., | Time | flux, | % of feed |
| Membrane | liquid | weight-% | h | g/m2/h | conductivity |
| Trisep TS40 | IEX water | 0 | 24 | 309 | 77 |
| Trisep TS40 | Ammonium | 5 | 24 | 389 | 80 |
| | hydroxide | | | | |
| Trisep TS40 | Ammonium | 10 | 24 | 393 | 80 |
| | hydroxide | | | | |

### Example 14

### Salt Flux Test after Treatment of GE Osmonics Desal 5 DL Membrane with Ammonium Hydroxide and Lactic Acid in Two Steps

A membrane treatment test was carried out with flat sheets. The nanofiltration membrane tested was GE Osmonics Desal 5 DL membrane. The filtration unit used in the test was Alfa Laval LabStak M20.

After the pre-washing steps in accordance with Example 1 (acetic acid soaking at 25° C. instead of 30° C.), the membrane sheets were treated by incubation in various test liquids at 25° C., 40° C. or 68° for 24 or 72 hours, followed by an optional second incubation according to Table 14. The test liquids were pure water, 40% lactic acid and 5% ammonium hydroxide, 5% Na₂CO₃ and 10% Na₂CO₃. After the soaking treatment, the membrane sheets were flushed well with ion free water before assembling them to the nanofiltration test unit.

A salt flux test with the treated membranes was carried by preparing 40 g/l lactose solution by dissolving lactose to ion free water. The lactose solution was also supplemented with 3 g/l NaCl and 0.4 g/l Na₂HPO₄. The pH of solution was adjusted with lactic acid to pH 5.5 The temperature of solution was adjusted to 25° C. and nanofiltration was started in reflux mode where the permeate is continuously fed back to the feed tank. The feed pressure was gradually raised to 15 bar and permeate flux was measured from each of the membrane. After the flux was stabilised (within about 30 minutes) samples were taken from the concentrate and permeate. The permeate flux values were registered and the permeate samples were analysed with a conductivity meter and with HPLC to measure the salt content and the lactose content to calculate the salt flux and the lactose flux. The membrane treatment methods, lactose and salt fluxes and salt retentions measured with respective membranes are presented in Table 14.

TABLE 14

| Treatment | | | | | | |
|---|---|---|---|---|---|---|
| liquid 1/ | | | | | | Salt retention, |
| temp./ | Treatment liquid | | Lactose | Lactose | | % of |
| treatment | 2/temp./ | Flux, | purity, | flux, | Salt flux, g | feed |
| time | treatment time | kg/m2/h | %/DS | g/m2/h | NaCl/m2/h | cond. |
| IEX water | — | 46 | 2.0 | 73 | 105 | 21 |
| (1) | | | | | | |
| 5% Ammonium | — | 64 | 0.6 | 30 | 167 | 10 |
| hydroxide, | | | | | | |
| 25° C., 72 h | | | | | | |
| Lactic acid | — | 50 | 1.4 | 42 | 120 | 17 |
| 40% Lactic | 5% Ammonium | 67 | 0.7 | 33 | 175 | 10 |
| acid, 68° C., | hydroxide, | | | | | |
| 40 h | 25° C., 5 h | | | | | |
| 40% Lactic | 5% Ammonium | 70 | 0.6 | 31 | 181 | 11 |
| acid, 68° C., | hydroxide, | | | | | |
| 72 h | 25° C., 24 h | | | | | |
| 5% Ammonium | 40% Lactic | 73 | 1.1 | 71 | 203 | 4 |
| hydroxide, | acid, 68° C., 24 h | | | | | |
| 25° C., 24 h | | | | | | |
| 5% Ammonium | 40% Lactic | 73 | 1.3 | 78 | 191 | 10 |
| hydroxide, | acid, 68° C., 24 h | | | | | |
| 25° C., 48 h | | | | | | |
| 5% Ammonium | 40% Lactic | 80 | 1.2 | 80 | 224 | 3 |
| hydroxide, | acid, 68° C., 24 h | | | | | |
| 25° C., 72 h | | | | | | |
| 5% NaCO₃, | — | 53 | 1.4 | 53 | 125 | 19 |
| 40° C., 24 h | | | | | | |
| 10% NaCO₃, | — | 52 | 1.2 | 47 | 122 | 19 |
| 40° C., | | | | | | |
| 24 h | | | | | | |
| 5% NaCO₃, | — | 57 | 1.1 | 45 | 136 | 17 |
| 40° C., 72 h | | | | | | |
| 10% NaCO₃, | — | 58 | 0.5 | 20 | 136 | 19 |
| 40° C., | | | | | | |
| 72 h | | | | | | |

1. A process of treating polymeric nanofiltration membranes before separation of low molecular weight compounds from a solution containing the same by nanofiltration, wherein the treatment of the nanofiltration membranes is performed with a treatment liquid under conditions which enhance the flux of the low molecular weight compounds to the nanofiltration permeate, wherein the treatment liquid contains one or more compounds selected from organic acids and alcohols, organic sulfonic acids and sulfonates, and surfactants.

2. The process as claimed in claim 1, wherein the treatment liquid contains one or more of organic acids, one or more of organic sulfonic acids and sulfonates, and one or more of surfactants.

3. The process as claimed in claim 1 or 2, wherein the organic acids are selected from formic acid, acetic acid, propionic acid, lactic acid, oxalic acid, citric acid, glycolic acid and aldonic acids.

4. The process as claimed in claim 3, wherein the alcohols are selected from methanol, ethanol, n-propanol, isopropanol and glycerol.

5. The process as claimed in any one of the preceding claims, wherein the organic sulfonic acids and sulfonates are selected from alkyl aryl sulfonic acids and sulfonates, taurine, perfluorooctane sulfonic acid and Nafion.

6. The process as claimed in claim 5, wherein the alkyl aryl sulfonic acids and sulfonates are selected from toluene sulfonic acid and sodium dodecylbenzenesulfonate.

7. The process as claimed in any one of the preceding claims, wherein the surfactants are selected from anionic tensides.

8. The process as claimed in any one of the preceding claims, wherein the surfactants are selected from cationic tensides.

9. The process as claimed in any one of the preceding claims, wherein the concentration of the compounds selected from organic acids and alcohols in the treatment liquid is in the range of 0.5% to 60%, preferably 0.5 to 20% and more preferably 0.5 to 10% by weight.

10. The process as claimed in any one of the preceding claims, wherein the concentration of the compounds selected from organic sulfonic acids and sulfonates in the treatment liquid is in the range of 0.1 to 10%, preferably 0.1 to 5% and more preferably 0.1 to 2% weight.

11. The process as claimed in any one of the preceding claims, wherein the concentration of the surfactants in the treatment liquid is in the range of 0.01 to 10%, preferably 0.01 to 5% and more preferably 0.01 to 2% by weight.

12. The process as claimed in claim 1, wherein the treatment liquid contains one or more of organic acids, one or more of organic sulfonic acids and one or more of anionic tensides.

13. The process as claimed in claim 12, wherein the organic acids comprise citric acid and lactic acid and the organic sulfonic acid is an alkyl aryl sulfonic acid.

14. A process of treating polymeric nanofiltration membranes before separation of low molecular weight compounds from a solution containing the same by nanofiltration, wherein the treatment of the nanofiltration membranes is performed with a treatment liquid under conditions which enhance the flux of the low molecular weight compounds to the nanofiltration permeate, wherein the treatment liquid contains one or more compounds selected from weak bases.

15. The process as claimed in claim 14, wherein the weak bases are selected from weak inorganic bases.

16. The process as claimed in claim 15, wherein the weak inorganic bases are selected from ammonium hydroxide, calcium hydroxide, magnesium hydroxide, sodium carbonate, calcium oxide and magnesium oxide.

17. The process as claimed in any one of claims 14 to 16, wherein the concentration of the weak bases in the treatment liquid is in the range of 0.5% to 60%, preferably 0.5 to 20% and more preferably 0.5 to 10% by weight.

18. The process as claimed in any one of the preceding claims, wherein the treatment is performed at a temperature of 20 to 100° C., preferably 20° C. to 90° C., more preferably 30° C. to 85° C., still more preferably 45 to 80° C. and especially 55 to 80° C.

19. The process as claimed in any one of claims 14 to 17, wherein the treatment is performed at a temperature of 20 to 40° C.

20. The process as claimed in any one of the preceding claims, wherein the treatment time is 0.5 to 150 hours, preferably 1 to 100 hours and more preferably 1 to 70 hours.

21. The process as claimed in any one of the preceding claims, wherein the treatment comprises two or more successive steps with different treatment liquids.

22. The process as claimed in claims 1, 14 and 21, wherein the treatment comprises at least one step with a treatment liquid containing one or more of weak inorganic bases and at least one step with a treatment liquid containing one or more of organic acids, in any desired sequence.

23. The process as claimed in claim 22, wherein the inorganic base is ammonium hydroxide and the organic acid is lactic acid.

24. The process as claimed in any one of the preceding claims, wherein the low molecular weight compounds have a molar mass of up to 360 g/mol.

25. The process as claimed in any one of the preceding claims, wherein the low molecular weight compounds are selected from sugars, sugar alcohols, inositols, betaine, glycerol, amino acids, uronic acids, carboxylic acids, aldonic acids and inorganic and organic salts.

26. The process as claimed in claim 25, wherein the sugars are monosaccharides.

27. The process as claimed in claim 26, wherein the monosaccharides are selected from pentoses and hexoses.

28. The process as claimed in claim 27, wherein the pentoses are selected from xylose and arabinose.

29. The process as claimed in claim 27, wherein the hexoses are selected from glucose, galactose, rhamnose, mannose, fructose, isomaltose and tagatose.

30. The process as claimed in claim 25, wherein the inorganic salts are selected from monovalent salts, preferably NaCl, NaHSO₄ and NaH₂PO₄.

31. The process as claimed in any one of the preceding claims, wherein the solution comprising low molecular weight compounds is selected from plant-based biomass hydrolysates and biomass extracts, starch hydrolysates, oligosaccharide-containing surups, glucose syryps, fructose syrups, maltose syrups, corn syrups and lactose-containing dairy products.

32. The process as claimed in any one of the preceding claims, wherein the polymeric nanofiltration membranes are polyamide membranes.

33. The process as claimed in claim 32, wherein the polyamide membranes are polypiperazineamide membranes.

34. The process as claimed in any one of the preceding claims, wherein the flux of the low molecular weight compounds to the nanofiltration permeate is in the range of 10 to 20 000 g/m²h.

35. The process as claimed in claim 34, wherein the flux of the sugars to the nanofiltration permeate is in the range of 20 to 15 000 g/m²h, preferably 100 to 8 000 g/m²h and more preferably 100 to 4 000 g/m²h.

36. The process as claimed in claim 34, wherein the flux of xylose to the nanofiltration permeate is in the range of 100 to 15 000 g/m²h, preferably 300 to 15 000 g/m²h and more preferably 1 000 to 15 000 g/m²h.

37. The process as claimed in claim 34, wherein the flux of glucose to the nanofiltration permeate is in the range of 200 to 15 000 g/m²h, preferably 200 to 10 000 g/m²h and more preferably 200 to 8 000 g/m²h.

38. The process as claimed in claim 34, wherein the flux of inorganic salts to the nanofiltration permeate is in the range of 20 to 2000 g/m²/h, preferably 40 to 1500 g/m²/h and more preferably 80 to 1000 g/m²/h.

39. The process as claimed in any one of the preceding claims, wherein the process further comprises nanofiltration of the solution comprising low molecular weight compounds to obtain a nanofiltration retentate and a nanofiltration permeate, whereby said low molecular weight compounds are separated into the nanofiltration permeate.

40. A process as claimed in claim 1 for separating and recovering xylose from a xylose-containing solution by nanofiltration with a polymeric nanofiltration membrane, comprising
treating the membrane with an treatment liquid comprising citric acid, lactic acid, an alkyl aryl sulfonic acid and anionic tensides in the following conditions:
concentration of citric acid 0.5 to 20% by weight,
concentration of lactic acid 0.5 to 20% by weight,
concentration of the alkyl aryl sulfonic acid 0.1 to 10% by weight,
concentration of the anionic tensides 0.1 to 10% by weight,
treatment temperature 50 to 70° C., and
treatment time 2 to 70 hours,
to obtain a treated nanofiltration membrane, followed by
nanofiltering the xylose-containing solution with the treated nanofiltration membrane with a xylose flux of 100 to 15 000 g xylose/m²h to the nanofiltration permeate, and
recovering xylose from the nanofiltration permeate.

41. A process as claimed in claim 1 for separating and recovering xylose from a xylose-containing solution by nanofiltration with a polymeric nanofiltration membrane, comprising, in any desired sequence
a step of treating the membrane with a treatment liquid containing lactic acid in the following conditions:
concentration of lactic acid 20 to 60% by weight,
treatment temperature 50 to 70° C., and
treatment time 2 to 80 hours, and
a step of treating the membrane with a treatment liquid containing ammonium hydroxide in the following conditions:
concentration of ammonium hydroxide 0.1 to 10% by weight,
treatment temperature 20 to 40° C.,
treatment time 2 to 80 hours,
to obtain a treated nanofiltration membrane, followed by
nanofiltering the xylose-containing solution with the treated nanofiltration membrane with a xylose flux of 100 to 15 000 g xylose/m²h to the nanofiltration permeate, and
recovering xylose from the nanofiltration permeate.
